(19) United States
(12) Patent Application Publication
(10) Pub. No.: US 2024/0116016 A1
(43) Pub. Date: Apr. 11, 2024

(54) DISSOLUTION SYSTEM

(71) Applicant: Cytiva US LLC, Marlborough, MA (US)

(72) Inventor: Arnaud DE BOULARD, Hoegaarden (BE)

(73) Assignee: Cytiva US LLC, Marlborough, MA (US)

(21) Appl. No.: 18/045,082

(22) Filed: Oct. 7, 2022

(51) Int. Cl. B01F 35/221; B01F 23/50; B01F 35/00; B01F 35/21; B01F 35/71

(52) U.S. Cl. CPC B01F 35/2211; B01F 23/56; B01F 35/187; B01F 35/2113; B01F 35/2132; B01F 35/7176; B01F 2101/22

(57) ABSTRACT

A dissolution system includes a dissolution line and a bypass line. The dissolution line fluidly couples a biocontainer, a pump, a cartridge, an upstream filter, and a downstream filter in a circulation loop. The bypass line is in fluid communication with the dissolution line at an upstream junction and a downstream junction such that the bypass line is in parallel relationship with the cartridge. A flow control system is configured to control the flow of fluid through the cartridge such that the flow of fluid through at least one of the dissolution line and the bypass line is selectively controlled based upon a pressure in the dissolution line downstream of the cartridge between the cartridge and the biocontainer.

### TECHNICAL FIELD

This patent disclosure relates generally to a dissolution system and, more particularly, to a dissolution system for preparing a buffer solution from a buffer powder dissolved in a liquid media.

### BACKGROUND

Buffer solutions are typically made in mixers at full strength and produced in large quantities for storage in totes. A buffer dissolution system typically includes a buffer powder that is loaded into a bag. The bag of buffer powder is often prepared in one room and then transported to another room for preparation of the buffer solution. The powder bag is raised above a mixer, often using a lift, then opened and emptied into the mixer, which mixes the emptied contents of the powder bag with the fluid at one time to provide a mixed solution. Next, the mixed solution is filtered with a sterile grade or bioburden reduction grade filter, and then the filtered and mixed solution is transported to a storage or transfer tank.

For a typical bioprocessing application, multiple buffer solutions are used with the demand for each buffer solution being variable and on the order of two thousand liters for each buffer in some situations. Because the square footage of bioprocessing facilities is at a premium, the totes are transported from the buffer preparation area to the process suite. With one room dedicated for biological preparation, another room for media and buffer preparation, and still another room for the process suite, a bioprocessing process operation can consume a substantial footprint. The sterility of the materials can be compromised at each transfer step. In addition, specialized moving equipment is sometimes required for the larger totes, and there are risks associated with operating such machinery and moving these loads.

There is a continued need in the art to provide additional solutions to enhance the management of buffer solutions used in various bioprocessing applications. For example, there is a continued need in the art to provide new and enhanced powder dissolution systems.

It will be appreciated that this background description has been created to aid the reader, and is not to be taken as an indication that any of the indicated problems were themselves appreciated in the art. While the described principles can, in some aspects and embodiments, alleviate the problems inherent in other systems, it will be appreciated that the scope of the protected innovation is defined by the attached claims, and not by the ability of any disclosed feature to solve any specific problem noted herein.

### BRIEF SUMMARY OF THE INVENTION

The present disclosure, in one aspect, is directed to embodiments of a dissolution system. In one embodiment, the dissolution system includes a biocontainer, a pump, a cartridge, an upstream filter, a downstream filter, and a dissolution line. The dissolution line fluidly couples the biocontainer, the pump, the cartridge, the upstream filter, and the downstream filter in a circulation loop.

The biocontainer defines a container inlet, a container outlet, and a storage volume. The container inlet and outlet are in communication with the storage volume. The storage volume is configured to hold a supply of fluid. The pump is in fluid communication with the storage volume of the biocontainer. The pump is adapted to receive the supply of fluid from the container outlet of the biocontainer and to discharge a flow of fluid therefrom in a circulation direction to the container inlet.

The cartridge defines a cartridge inlet, a cartridge outlet, and a storage chamber. The cartridge inlet and outlet are in communication with the storage chamber. The storage chamber is configured to hold an amount of solute for dissolution into the supply of fluid. The storage chamber is in fluid communication with the pump via the cartridge inlet to receive the flow of fluid therefrom. The cartridge inlet, the storage chamber, and the cartridge outlet are configured such that the flow of fluid is directed from the cartridge inlet through storage chamber and out the cartridge outlet to flow past the amount of solute in the storage chamber. The cartridge outlet is in fluid communication with the container inlet.

The upstream filter is in fluid communication with the biocontainer and the cartridge such that the upstream filter is interposed between the container outlet of the biocontainer and the cartridge inlet of the cartridge upstream of the cartridge relative to the circulation direction. The downstream filter is in fluid communication with the cartridge and the biocontainer such that the downstream filter is interposed between the cartridge outlet of the cartridge and the container inlet of the biocontainer downstream of the cartridge relative to the circulation direction.

In another embodiment, the dissolution system includes a dissolution line, which fluidly couples a biocontainer, a pump, a cartridge, an upstream filter, and a downstream filter in a circulation loop, a bypass line, and means for controlling the flow of fluid through the cartridge. The biocontainer is configured to hold a supply of fluid. The pump is adapted to receive the supply of fluid from the biocontainer and to discharge a flow of fluid therefrom in a circulation direction. The cartridge is configured to hold an amount of solute for dissolution into the supply of fluid. The cartridge is in fluid communication with the pump to receive the flow of fluid therefrom and to pass the flow of fluid therethrough. The upstream filter is in fluid communication with the biocontainer and the cartridge such that the upstream filter is interposed between the biocontainer and the cartridge upstream of the cartridge relative to the circulation direction, and the downstream filter is in fluid communication with the cartridge and the biocontainer such that the downstream filter is interposed between the cartridge and the biocontainer downstream of the cartridge relative to the circulation direction.

The dissolution line includes an upstream junction and a downstream junction. The upstream junction is disposed upstream of the cartridge relative to the circulation direction between the upstream filter and the cartridge, and the downstream junction is disposed downstream of the cartridge relative to the circulation direction between the cartridge and the downstream filter. The bypass line is in fluid communication with the dissolution line at the upstream junction and the downstream junction such that the bypass line is in parallel relationship with the cartridge. The means for controlling the flow of fluid through the cartridge are configured to selectively control the flow of fluid through at least one of the dissolution line and the bypass line based upon a pressure in the dissolution line downstream of the cartridge between the cartridge and the biocontainer.

In another aspect, the present disclosure is directed to embodiments of a method for preparing a buffer dissolution. In one embodiment, a method of preparing a buffer solution includes fluidly coupling a buffer cartridge in a circulation loop formed by a dissolution line. The dissolution line fluidly couples in the circulation loop a biocontainer, a pump adapted to discharge a flow of fluid therefrom in a circulation direction, the buffer cartridge, an upstream filter disposed upstream of the buffer cartridge relative to the circulation direction between the biocontainer and the buffer cartridge, and a downstream filter disposed upstream of the buffer cartridge relative to the circulation direction between the buffer cartridge and the biocontainer. The buffer cartridge contains an amount of a buffer solute. A flow of fluid is circulated through the circulation loop to entrain at least a portion of the buffer solute from the buffer cartridge into the flow of fluid.

Further and alternative aspects and features of the disclosed principles will be appreciated from the following detailed description and the accompanying drawings. As will be appreciated, the dissolutions systems and the methods of preparing a solution disclosed herein are capable of being carried out in other and different embodiments, and capable of being modified in various respects. Accordingly, it is to be understood that both the foregoing general description and the following detailed description are exemplary and explanatory only and do not restrict the scope of the appended claims

### BRIEF DESCRIPTION OF THE SEVERAL VIEWS OF THE DRAWINGS

FIG. 1 is a schematic view of an embodiment of a dissolution system constructed in accordance with principles of the present disclosure.

FIG. 2 is a schematic view of an embodiment of a dissolution system and process constructed and performed in accordance with principles of the present disclosure.

FIG. 3 is a schematic view of an embodiment of a dissolution system constructed in accordance with principles of the present disclosure.

FIG. 4 illustrates an embodiment of a controller processing system usable with embodiments of the dissolution system and process according to principles of the present disclosure.

It should be understood that the drawings are not necessarily to scale and that the disclosed embodiments are illustrated diagrammatically and in partial views. In certain instances, details which are not necessary for an understanding of this disclosure or which render other details difficult to perceive may have been omitted. It should be understood that this disclosure is not limited to the particular embodiments illustrated herein.

### DETAILED DESCRIPTION OF THE INVENTION

Embodiments of a dissolution system constructed in accordance with principles of the present disclosure can be adapted to be used with embodiments of a method of preparing a solution performed in accordance with principles of the present disclosure. Embodiments of a dissolution system constructed in accordance with principles of the present disclosure can be used in biopharmaceutical environments, but can also be used in other applications, e.g., where different solutions, powders, biologicals, fluids, reagents and/or chemicals are used for preparations.

Embodiments of a dissolution system constructed in accordance with principles of the present disclosure can be used to enable the direct dissolution and sterilization of powder by recirculation, and to simplify the dilution of powders in a bioprocess, such as a suitable buffer powder, for example. For example, a buffer preparation process following principles of the present disclosure can be used in a process of manufacturing biopharmaceuticals, such as, antibodies, for example. One skilled in the art will appreciate other uses for a system and a method following principles of the present disclosure that are suitable in settings in which effective solute dissolution is desired, such as, media preparation where the subsequent process benefits from a sterile addition of any of the final buffer or media. The present disclosure can also find uses outside of the biopharmaceutical area, such as for the production of a saline, a common need of hospitals.

Embodiments of a dissolution system constructed in accordance with principles of the present disclosure can more easily maintain the sterility of a biocontainer during the dissolution process even when recirculating with a non-sterile powder container or buffer cartridge, while at the same time reducing the number of steps and the amount of time necessary to complete the dissolution of the solute into the solvent by utilizing an effective solute distribution.

In embodiments, a dissolution line of a dissolution system constructed according to principles of the present disclosure can be operated to provide closed loop recirculation of the solvent through an amount of solute stored in a cartridge. The closed loop arrangement can improve operation by reducing exposure to external contaminants, such as an air interface of the mixing process, further improving sterility confidence. Additionally, the inclusion of a bypass line in embodiments of a dissolution system constructed according to principles of the present disclosure can help prevent too much solute from accumulating in any one component of the system, e.g., a filter, a pre-filter, etc.

Still further, embodiments of a dissolution system constructed according to principles of the present disclosure can help improve industrial and manufacturing aspects. For example, in embodiments, buffer solution can be prepared on site, e.g., in the same room as the buffer storage or at the final use location. In embodiments, the preparation of the buffer solution at or near the point of use can streamline the application and enables the combination of mixing, sterile filtration, and liquid storage in one process step, thereby reducing the footprint of the buffer preparation process. Substocks of materials and materials transportation logistics can be reduced and used to save on bioprocessing facilities space. Not only does the reduction in footprint save costs, but the quicker production time, capability of instant batch release, and smaller collection of components relative to current preparation processes reduces costs of the process itself.

In embodiments of a dissolution system constructed in accordance with principles of the present disclosure, the dissolution system include a biocontainer, a pump, a cartridge, an upstream filter, a downstream filter, and a dissolution line. The dissolution line fluidly couples the biocontainer, the pump, the cartridge, the upstream filter, and the downstream filter in a circulation loop.

The biocontainer defines a container inlet, a container outlet, and a storage volume. The container inlet and outlet are in communication with the storage volume. The storage volume is configured to hold a supply of fluid. The pump is in fluid communication with the storage volume of the biocontainer. The pump is adapted to receive the supply of fluid from the container outlet of the biocontainer and to discharge a flow of fluid therefrom in a circulation direction to the container inlet.

The cartridge defines a cartridge inlet, a cartridge outlet, and a storage chamber. The cartridge inlet and outlet are in communication with the storage chamber. The storage chamber is configured to hold an amount of solute for dissolution into the supply of fluid. In embodiments, any suitable solute can be used, such as any suitable solid. In embodiments, the solute can take any suitable form, such as powder or pellet form, for example.

The storage chamber is in fluid communication with the pump via the cartridge inlet to receive the flow of fluid therefrom. The cartridge inlet, the storage chamber, and the cartridge outlet are configured such that the flow of fluid is directed from the cartridge inlet through storage chamber and out the cartridge outlet to flow past the amount of solute in the storage chamber. The cartridge outlet is in fluid communication with the container inlet.

The upstream filter is in fluid communication with the biocontainer and the cartridge such that the upstream filter is interposed between the container outlet of the biocontainer and the cartridge inlet of the cartridge upstream of the cartridge relative to the circulation direction. The downstream filter is in fluid communication with the cartridge and the biocontainer such that the downstream filter is interposed between the cartridge outlet of the cartridge and the container inlet of the biocontainer downstream of the cartridge relative to the circulation direction.

In embodiments, a dissolution system constructed in accordance with principles of the present disclosure can include a valve adapted to selectively control the flow of fluid through the cartridge. In embodiments, a controller is provided that is configured to control the valve based upon a pressure in the dissolution line downstream of the cartridge between the cartridge and the biocontainer. In embodiments, a pressure sensor is disposed in the dissolution line and is in operable arrangement with the controller to transmit a pressure signal indicative of the pressure sensed by the sensor in the dissolution line.

In embodiments, a dissolution system constructed in accordance with principles of the present disclosure can include a bypass line in fluid communication with the dissolution line at an upstream junction and a downstream junction such that the bypass line is in parallel relationship with the cartridge. The dissolution system can include a flow control system configured to selectively control the flow of fluid through at least one of the dissolution line and the bypass line.

In embodiments, a dissolution system constructed in accordance with principles of the present disclosure can include a pre-filter in fluid communication with the cartridge and the downstream filter such that the pre-filter is interposed between the cartridge and the downstream filter. The pre-filter can have an interior volume which is greater than the interior volume of the downstream filter.

In embodiments, a dissolution system constructed in accordance with principles of the present disclosure can include a mixer in fluid communication with the cartridge and the downstream filter such that the mixer is interposed between the buffer cartridge and the downstream filter. In embodiments, the mixer can be any suitable mixer, such as, for example, a static mixer.

In embodiments, a dissolution system constructed in accordance with principles of the present disclosure can include a solution characteristic sensor disposed in the dissolution line. The solution characteristic sensor is configured to generate a characteristic signal corresponding to a value of a solution characteristic sensed in the dissolution line by the solution characteristic sensor. At least one of a dissolution valve and a bypass valve can be adapted to operate based upon the pressure signal.

In embodiments, the solution characteristic sensor can comprise any suitable sensor, such as a suitable pH sensor, for example, which generates a pH signal indicative of the pH sensed by the pH sensor. In embodiments, the dissolution system can include a pH adjustment line and a pH adjustment valve. The pH inlet line is in fluid communication with the dissolution line and is adapted to deliver a supply of pH-adjusting fluid to the dissolution line. The pH adjustment valve is operable to selectively occlude the pH adjustment line to interrupt the flow of the supply of pH-adjusting fluid to the dissolution line. The pH adjustment valve is adapted to operate based upon the pH signal via control by a suitable controller.

In embodiments, a dissolution system constructed in accordance with principles of the present disclosure can include means for controlling the flow of fluid through the cartridge. The flow controlling means can be configured to selectively control the flow of fluid through at least one of the dissolution line and the bypass line based upon a pressure in the dissolution line downstream of the cartridge between the cartridge and the biocontainer. In embodiments, the flow controlling means can include a dissolution pump disposed in the dissolution line between the upstream junction and the cartridge inlet, a bypass pump disposed in the bypass line, a pressure sensor disposed in the dissolution line between the cartridge outlet and the container inlet, and a controller in operable arrangement with the pressure sensor, the dissolution pump, and the bypass pump. The pressure sensor is configured to generate a pressure signal corresponding to a pressure sensed in the bypass line by the pressure sensor and to transmit the pressure signal to the controller. The controller is configured to operate at least one of the bypass pump and the dissolution pump based upon the pressure signal.

In embodiments, a dissolution system constructed in accordance with principles of the present disclosure can include means for controlling the flow of fluid through the cartridge that includes a pump disposed in the dissolution line between the cartridge outlet and the upstream junction, a dissolution valve, a bypass valve, a pressure sensor, and a controller. The dissolution valve is disposed in the dissolution line between the upstream junction and the cartridge inlet, the bypass valve is disposed in the bypass line, and the pressure sensor is disposed in the dissolution line between the cartridge outlet and the container inlet. The pressure sensor is configured to generate a pressure signal corresponding to a pressure sensed in the dissolution line by the pressure sensor and to transmit the pressure signal to the controller. The controller is configured to operate at least one of the dissolution valve, the bypass valve, and the pump based upon the pressure signal.

In embodiments, a method of preparing a solution following principles of the present disclosure comprises using a using a recirculation system with a dissolution line, a bypass line, and a flow control system following principles of the present disclosure. In embodiments, a recirculation system constructed according to principles of the present disclosure includes a storage biocontainer and a solids cartridge in fluid communication with each other via a dissolution line, a bypass line is in fluid communication with the dissolution such that the bypass line is in parallel relationship with the cartridge, and a flow control system configured to selectively direct flow through either the cartridge or the bypass line.

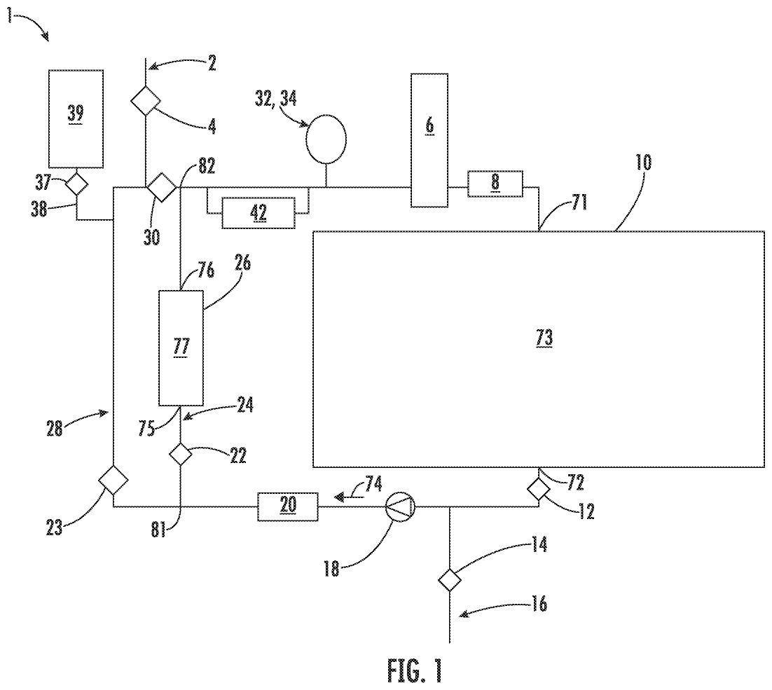

Turning now to the Figures, there is shown in FIG. 1 an embodiment of a buffer dissolution system 1 constructed according to principles of the present disclosure. In embodiments, the system 1 comprises a single use system.

In embodiments, the system 1 can be sterilized using any suitable technique. For example, in embodiments, gamma or x-ray irradiation of the system 1 can occur to sterilize it. In other embodiments, other ways of sterilizing the system 1 can be used, such as ozonation or high pressure air/water (steam) sterilization.

The illustrated dissolution system 1 includes a biocontainer 10, a pump 18, an upstream filter 20, a cartridge 26, a mixer 42, a pre-filter 6, a downstream filter 8, a dissolution line 24, a bypass line 28, and means 22, 23 for controlling the flow of fluid through the cartridge 26 by selectively directing a flow of fluid through the cartridge 26 and the bypass line 28. The dissolution line 24 fluidly couples the biocontainer 10, the pump 18, the upstream filter 20, the cartridge 26, and the downstream filter 8 in a circulation loop 69.

The biocontainer 10 is configured to hold a supply of fluid. In embodiments, the biocontainer 10 can be any suitable storage vessel configured to hold a desired volume of liquid solution, such as a commercially-available “2D” (or “two-dimensional”) biocontainer bag or “3D” (three-dimensional) biocontainer tank. The biocontainer 10 can be a rigid tank or bag, or a more flexible container. For example, the biocontainer 10 can be made of a low density polypropylene and polyethylene mixture.

The biocontainer 10 defines a container inlet 71, a container outlet 72, and a storage volume 73. The container inlet 71 and the container outlet 72 are in communication with the storage volume 73. The storage volume 73 is configured to hold the supply of fluid.

The pump 18 is in fluid communication with the storage volume 73 of the biocontainer 10. The pump 18 is adapted to receive the supply of fluid from the container outlet 72 of the biocontainer 10 and to discharge a flow of fluid therefrom in a circulation direction 74 to the container inlet.

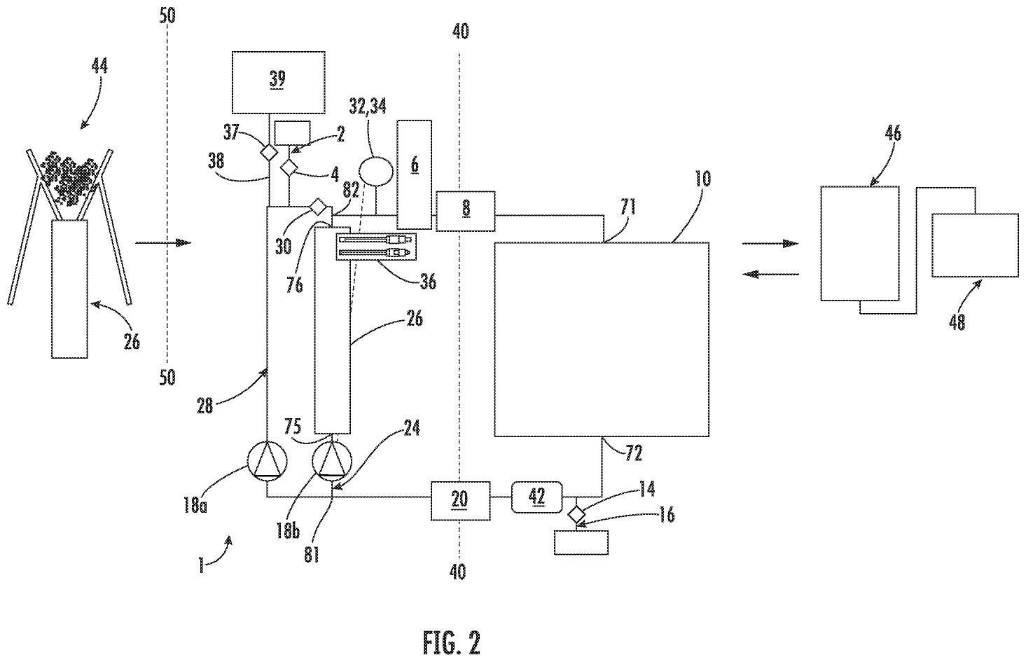

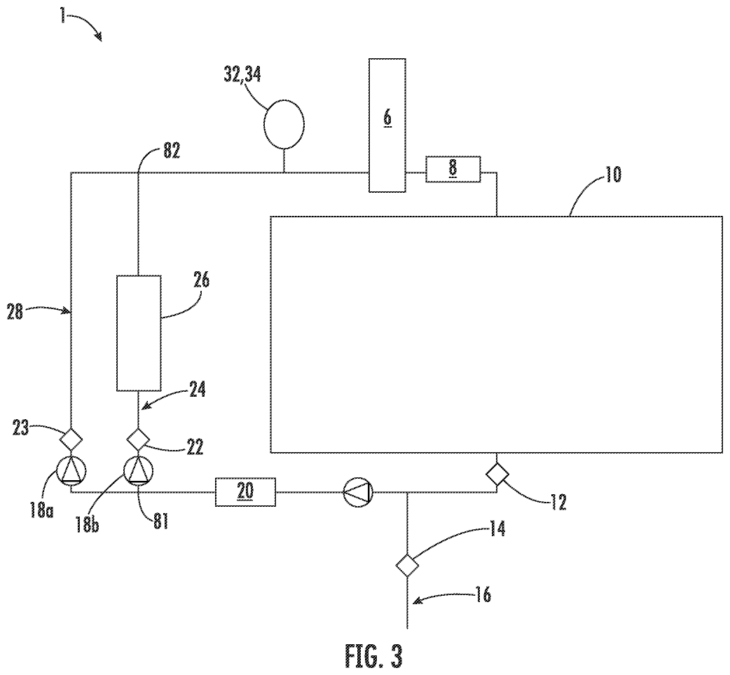

In embodiments, the pump 18 can be any suitable pump, as will be appreciated by one skilled in the art. For example, in embodiments, the pump 18 can be a suitable peristaltic pump or a suitable variable displacement pump, for example. While in the embodiment of FIG. 1 the pump 18 is shown as being disposed downstream of the outlet line 16, the location of the pump 18 can change relative to the other components of the system 1, such as is shown in the embodiments of FIGS. 2 and 3, for example.

The cartridge 26 is configured to hold an amount of solute for dissolution into the supply of fluid. The cartridge 26 is in fluid communication with the pump 18 to receive the flow of fluid therefrom and to pass the flow of fluid therethrough.

The cartridge 26 defines a cartridge inlet 75, a cartridge outlet 76, and a storage chamber 77. The cartridge inlet 75 and the cartridge outlet 76 are in communication with the storage chamber 77. The storage chamber 77 is configured to hold an amount of solute for dissolution into the supply of fluid. The storage chamber 77 is in fluid communication with the pump 18 via the cartridge inlet 75 to receive the flow of fluid therefrom. The cartridge inlet 75, the storage chamber 77, and the cartridge outlet 76 are configured such that the flow of fluid is directed from the cartridge inlet 75 through storage chamber 77 and out the cartridge outlet 76 to flow past the amount of solute in the storage chamber 77. The cartridge outlet 76 is in fluid communication with the container inlet 71.

The upstream filter 20 is in fluid communication with the biocontainer 10 and the cartridge 26 such that the upstream filter 20 is interposed between the container outlet 72 of the biocontainer 10 and the cartridge inlet 75 of the cartridge 26 upstream of the cartridge 26 relative to the circulation direction 74.

The downstream filter 8 is in fluid communication with the cartridge 26 and the biocontainer 10 such that the downstream filter 8 is interposed between the cartridge outlet 76 of the cartridge 26 and the container inlet 71 of the biocontainer 10 downstream of the cartridge 26 relative to the circulation direction 74.

The pre-filter 6 is in fluid communication with the cartridge 26 and the downstream filter 8 such that the pre-filter 6 is interposed between the cartridge 26 and the downstream filter 8. In embodiments, the pre-filter 6 has an interior volume that is greater that the interior volume of the downstream filter 8. The pre-filter 6 and the upstream and downstream filters 20, 8 can comprise any suitable filter including any suitable filter membrane, such as, a filter including a high density polyethylene housing and a filter membrane made out of polyethylene or polypropylene, for example.

The mixer 42 is in fluid communication with the cartridge 26 and the downstream filter 8 such that the mixer 42 is interposed between the cartridge 26 and the downstream filter 8. In embodiments, the mixer 42 can be any suitable mixer, such as, e.g., a static mixer.

In embodiments, the buffer cartridge 26 is in fluid communication with the pre-filter 6, the downstream filter 8, the static mixer 42, and the pressure sensor 34. The downstream filter 8 in the embodiment of FIG. 1 is preceded in the circulation direction 74 by the pre-filter 6, where the pre-filter 6 has a larger volume than the downstream filter 8.

The dissolution line 24 includes an upstream junction 81 and a downstream junction 82. The upstream junction 81 is disposed upstream of the cartridge 26 relative to the circulation direction 74 between the upstream filter 20 and the cartridge 26. The downstream junction 82 is disposed downstream of the cartridge 26 relative to the circulation direction 74 between the cartridge 26 and the downstream filter 8. The bypass line 28 is in fluid communication with the dissolution line 24 at the upstream junction 81 and the downstream junction 82 such that the bypass line 28 is in parallel relationship with the cartridge 26.

The means 22, 23 for controlling the flow of fluid through the cartridge 26 are configured to selectively control the flow of fluid through at least one of the dissolution line 24 and the bypass line 28 based upon a pressure in the dissolution line 24 downstream of the cartridge 26 between the cartridge 26 and the biocontainer 10. In embodiments, the flow control means comprises a suitable flow control system configured to selectively control the flow of fluid through the dissolution line 24 and the bypass line 28 based upon the pressure in the dissolution line 24 downstream of the cartridge 26 between the cartridge 26 and the biocontainer 10. A pressure sensor 34 can be used to detect the pressure in the dissolution line 24 downstream of the cartridge 26 between the cartridge 26 and the biocontainer 10.

The pump 18 is disposed in the dissolution line 24 between the container outlet 72 of the biocontainer 10 and the upstream junction 81. In the embodiment illustrated in FIG. 1, the flow control means comprises a flow control system including a dissolution valve 22, a bypass valve 23, and the pressure sensor 34. The dissolution valve 22 is disposed in the dissolution line 24 between the upstream junction 81 and the cartridge inlet 75. The bypass valve 23 is disposed in the bypass line 28. The pressure sensor 34 is disposed in the dissolution line 24 between the cartridge outlet 76 and the container inlet 71 of the biocontainer 10. The pressure sensor 34 is configured to generate a pressure signal corresponding to a pressure sensed in the dissolution line 24 by the pressure sensor 34. In embodiments, the pressure sensor 34 is in operable communication with a suitable controller configured to operate at least one of the dissolution valve 22 and the bypass valve 23 based upon the pressure signal. For example, when the pressure is above a predetermined value, the controller can be configured to open the bypass valve 23 so that fluid is diverted from the dissolution line 24 to the bypass line 28. In embodiments, the controller can be configured to independently vary the position of each of the dissolution valve 22 and the bypass valve 23 between a fully open position and a fully closed position to maintain the pressure in the dissolution line 24 as sensed by the pressure sensor 34 within a predetermined range.

In embodiments, a solution characteristic sensor 32 is disposed in the dissolution line 24. The solution characteristic sensor 32 can be configured to generate a characteristic signal corresponding to a value of a solution characteristic sensed in the dissolution line 24 by the solution characteristic sensor 32. In the illustrated embodiment, the solution characteristic sensor comprises a pH sensor, and the characteristic signal comprises a pH signal.

The system 1 includes a pH adjustment line 38 and a pH adjustment valve 37. The pH adjustment line 38 is in fluid communication with the dissolution line 24 and is adapted to deliver a supply of pH-adjusting fluid 39 to the dissolution line 24. The pH adjustment valve 37 is operable to selectively occlude the pH adjustment line 38 to interrupt the flow of the supply of pH-adjusting fluid 39 to the dissolution line 24. The pH adjustment valve 37 is adapted to operate based upon the pH signal generated by the pH sensor 32. In the illustrated embodiment, the pH adjustment line 38 is in fluid communication with the dissolution line 24 via the bypass line 28.

Referring to FIG. 2, another embodiment of a dissolution system 201 constructed according to principles of the present disclosure system includes flow control means comprising a flow control system configured to selectively control the flow of fluid through the dissolution line 24 and the bypass line 28 including a bypass pump 18a disposed in the bypass line 28, a dissolution pump 18b disposed in the dissolution line 24 between the upstream junction 81 and the cartridge inlet 75, and the pressure sensor 34. The pressure sensor 34 is disposed in the dissolution line 24 between the cartridge outlet 76 and the container inlet 71 of the biocontainer 10. The pressure sensor 34 is configured to generate a pressure signal corresponding to a pressure sensed in the dissolution line 24 by the pressure sensor 34. In embodiments, at least one of the bypass pump 18a and the dissolution pump 18b is adapted to operate based upon the pressure signal.

As shown in FIG. 2, the buffer cartridge 26 can be filled with an amount of solid material 44, such as any suitable solute, for dispersion in a fluid. In embodiments, the solute 44 can comprise any suitable solid for dissolution in a fluid, such as a suitable powder or pellets to be dissolved in a solvent. For example, in embodiments, the solute 44 can comprise a buffer salt powder, for example, or other suitable powder. In embodiments, the solute 44 may constitute a minority or majority of the concentration of the end solution. The buffer cartridge 26 may retain the solid 44 in a storage area or chamber, and that storage area or chamber, which can define a specific, fixed, or adjustable volume, may be put in fluid communication with the dissolution line 24 of the system 1. The buffer cartridge 26 can be filled with the solid material 44 in one location and then transferred to another location, e.g., a final use location or a buffer preparation location, where a location division line 50 of FIG. 2 schematically represents the division of the filling location and the other location. In embodiments, the buffer cartridge 26 can be filled in the same location as the system 1 without requiring an additional location.

In embodiments, the buffer cartridge 26 can comprise a single use container for the solid material to be dispersed in the fluid and introduced to the system 1 as needed. The buffer cartridge 26 can be rigid, semi-rigid, or any amount of rigidity that is able to withstand a sufficient amount of pressure for its intended application. In embodiments, the buffer cartridge 26 can be configured to house a predetermined amount of a solid for dissolution in fluid. In embodiments, the system 1 can include a plurality of buffer cartridges 26 in series fluid communication with each other for applications in which the amount of solid material to be dissolved is greater than the amount held by a single cartridge 26. In embodiments, the buffer cartridge 26 can be sterilized using any suitable technique as will be appreciated by one skilled in the art.

The flow paths and lines of the system 1 can be made of any suitable material, e.g., silicon tubing, thermoplastic polyethylene or polypropylene. Any suitable valve can be used to selectively occlude a flow path and/or line of the system. In embodiments, in which the lines are made from a resiliently flexible material, a suitable clamp (or pinch) valve can be used. While the fluid (solvent) is typically water, the fluid can, in embodiments, also include or be other liquids and solutions such as organic solvents or mixtures of organic solvents with water (e.g. ethanol and water).

Referring to FIG. 1, an inlet line 2 allows for fluid to enter and be introduced to the system 1 when a clamp valve 4 is open. When the clamp valve 4 is closed, no further fluid is introduced to the system 1 from the source of the inlet line 2. Closure of the clamp valve 4 can help to ensure that once a specific volume of fluid has been provided to the system 1, the amount of fluid in the system 1 will not change, thereby helping to create a closed loop circulation circuit.

Fluid can flow from the inlet line 2 through the dissolution line 24 in the circulation direction 74 via operation of the pump 18. As the fluid initially flows through the dissolution line 24, the circulating fluid can vent components of the system 1, such as, the pre-filter 6 and the sterile upstream and downstream filters 20, 8.

Fluid can fill the biocontainer 10 by flowing through the inlet 71 of the biocontainer 10 before, while, or after venting the pre-filter 6 and the upstream and downstream filters 20, 8. If either a closeable outlet 72 of the biocontainer 10 or the clamp valve 12 is closed, the biocontainer 10 can be filled to a desired volume before the fluid flows through the clamp valve 12 to the other components of the system 1. In embodiments, the biocontainer 10 can be filled with fluid and the filled volume measured or determined via suitable means such as weight measurement with a scale or flow measurement with a suitable flow gauge. Filling the biocontainer 10 can occur with or without the cartridge 26 in the system 1 depending on the positioning and open or closed status of the valves 12, 22, 23 of the system 1. Once a sufficient volume or amount of fluid has been introduced to the system 1, the connection to the source of fluid can be removed from the open connection(s) in the dissolution line 24 and the buffer cartridge 26 can be introduced to the dissolution line 24.

An outlet line 16 can be provided to facilitate the draining of the fluid in the system 1. The clamp valve 14 of the outlet line 16 can be kept closed such that fluid can circulate through the closed loop formed by the dissolution line 24 and the bypass line 28. However, the initial flow into the system 1 may be aided by keeping the clamp valve 14 open so that the fluid can flow through the outlet line 16 and flush out various components of the system 1, carrying whatever particulates the fluid picked up on the way through the system 1. In embodiments, the outlet line 16 can include an additional, sterile filter for further fluid processing or a sterile disconnector. Additionally, the clamp valve 14, can be any suitable valve. In embodiments, a selective filter can be provided in addition to, or in lieu of, the valve 14 that directs part of the fluid to the outlet line 16 and keeps part of the fluid in the system 1 based on any number of criteria, such as average particulate size or fluid pressure levels exerted on the line at the location of the clamp valve 14.

In embodiments, the biocontainer 10 is in fluid communication with the upstream filter 20, and the upstream filter 20 is in fluid communication with the bypass line 28 and the dissolution line 24. From the biocontainer 10, the fluid can flow through the upstream filter 20 and into the dissolution line 24 if the clamp valve 22 is at least partially open and into the bypass line 28 if the clamp valve 23 is at least partially open.

In embodiments, the bypass line 28 may have a larger cross-sectional area than the dissolution line 24, or be made of different material than the buffer cartridge 26 such that it is able to carry more fluid or support a larger fluid pressure. For the initial flow of fluid, the clamp valve 22 can be kept closed and fluid can flow through the bypass line 28. The fluid can flow through the bypass line 28 and through the clamp valve 30. The flow of fluid can recirculate in this circulation loop as many times as desired. In embodiments, the flow of fluid is not circulated through the bypass line 28 at startup, and circulation through the cartridge 26 to cause buffer powder dissolution can begin with startup. As a result, fluid can be selectively diverted into the bypass line 28 as desired. As mentioned above, the clamp valves 4, 12, 14, 23, 30 can be opened and closed at any time, and to any degree, in the initial flow circulation as desired to increase, decrease, or exchange fluid in the system 1, manage circulation of the fluid, or to manage fluid pressure and volume in any specific component, such as the biocontainer 10, as will be readily understood by one skilled in the art.

In embodiments, the buffer cartridge 26 is in fluid communication with the dissolution line 24. Once the clamp valve 22 is at least partially open, the fluid can flow through the dissolution line 24 and into the buffer cartridge 26. The buffer cartridge 26 can contain any number of solid materials 44, such as substances that are soluble into the fluid. In the embodiment of FIG. 1, the solid material 44 has a powder form, and the cartridge 26 contains a certain volume of buffer solute powder to be dissolved into the fluid and create the buffer preparation. The fluid can then flow into and out of the cartridge 26, picking up and carrying out buffer solute powder.

As the fluid flows through the buffer cartridge 26, the fluid picks up a portion of the substances 44 of solute powder in the buffer cartridge 26 and carries the solute powder with it to the pre-filter 6, or to the static mixer 42. After the fluid exits the buffer cartridge 26 in the embodiment of FIG. 2 and/or the static mixer 42 as in the embodiment of FIG. 1, the fluid can flow through the pre-filter 6 and then the downstream filter 8. The larger volume of the pre-filter 6 can help facilitate a better local dissolution of the solute powder before entering the downstream filter 8.

In the embodiment of FIG. 1, the cartridge 26 has an inlet 75 and a separate outlet 76 such that the fluid can flow directly through the buffer cartridge 26 in the path of the dissolution line 24, which can help to reduce turbulence in the buffer cartridge 26 and facilitate effective dispersal of buffer solute powder from the buffer cartridge 26. The inlet 75 and the outlet 76 can include fluid connectors in conjunction with clamp valves, or separate connectors such as snap or threaded connectors. The shape and size of the buffer cartridge 26 or components of the buffer cartridge 26 can vary as needed, and can be advantageously adapted to reduce risk of buffer solute loss, damage, or contamination when being filled or connected. Moreover, a mesh can be installed at the inlet 75 or the outlet 76 of the buffer cartridge 26 if desired to reduce the risk of a large quantity of powder exiting the container and delivering too much solute into circulation at one time.

In other embodiments, the buffer cartridge 26 may have one large opening on the bottom, top, or side of the buffer cartridge 26 that acts as both the inlet and outlet with a mesh at the interface of the buffer cartridge 26. The buffer cartridge 26 may also include an internal analysis sensor 36 such as a pH sensor or conductivity sensor, as shown in FIG. 2. An index of refractivity sensor can also be utilized as an analysis sensor 36, and installed following the biocontainer 10 but preceding the upstream filter 8.

Embodiments of the present disclosure may utilize a pre-filter 6 and a downstream filter 8 in any desired relative size proportion to manage conditions of the system 1, e.g., pressure or the flow of fluid. Alternatively, the downstream filter 8 can have a large volume or capacity, or have both a pre-filter chamber and a sterile chamber, increasing the local dissolution and allowing the downstream filter 8 to operate with or without the pre-filter 6 preceding the downstream filter 8. An additional pressure sensor similar to the pressure sensor 34 can be placed after the downstream filter 8. The information from the additional pressure sensor can provide an indication of the pressure drop through the pre-filter 6 and the downstream filter 8, and can indicate the dissolution level of the fluid in circulation.

In embodiments, the downstream filter 8 is in fluid communication with the storage chamber of the biocontainer 10. The downstream filter 8 can take the non-sterile fluid flowing out of the buffer cartridge 26 and ensure the fluid containing the entrained solute powder that flows into the biocontainer 10 is sterile before entering the biocontainer 10. The sterile upstream and downstream filters 20, 8 can be any suitable sterile barrier facilitating devices, such as filter discs, capsules, or cartridges, and can be any suitable size. After the downstream filter 8 filters the fluid containing solute to ensure sterility, the fluid and the solute flow into the biocontainer 10.

If the biocontainer 10 acts as a reservoir, the fluid and solute will achieve a distribution of solute powder proportional to the amount of solute by volume in the fluid. Moreover, in embodiments, the biocontainer 10 inlets and outlets can be configured and placed to promote mixing. In embodiments, the biocontainer 10 is in fluid communication with the upstream filter 8, and the fluid flows from the biocontainer 10 into the upstream filter 8. Fluid may flow from the biocontainer 10 solely by the pressure and flow generated by the pump 18, or may utilize other methods in combination with the pump 18 to assist flow, such as positioning the upstream filter 8 below or above the biocontainer 10 to increase or decrease the speed of the fluid into the upstream filter 8 or to increase or decrease the amount or likelihood of fluid that flows back into the biocontainer 10 after leaving the biocontainer 10 but before entering the upstream filter 8.

After fluid flows from the upstream filter 8, the fluid continues to recirculate throughout the system 1 and deposit an increasing amount of buffer solute in the biocontainer 10. At some point, a desired buffer preparation is achieved in the biocontainer 10. In some embodiments, the fluid gradually pulls more solute powder from the buffer cartridge 26 with each circulation until the proportion of solute to solvent is the same inside the buffer cartridge 26 as in the biocontainer 10. In another embodiment, the fluid circulates until the buffer cartridge 26 is emptied of solute powder and the fluid circulating through the biocontainer 10 possesses all desired solute powder. In some embodiments, the fluid pressure and flow rate is gradually increased over many circulation cycles to facilitate effective release of solute powder and manage fluid pressure in the system 1. Once the desired preparation is achieved, the buffer preparation can be removed for use as a buffer solution 46 in a desired application, such as a tangential flow filtration (TFF) or chromatography process 48, as shown in the embodiment of FIG. 2.

Referring to FIG. 2, to help regulate or determine the conditions of the fluid in the biocontainer 10 or flowing through the dissolution line 24 and/or the bypass line 28, at least one analytical sensor 36, such as, a pH sensor, conductivity sensor, or index of refraction sensor, for example, can be employed along with some components able to be used in response to the analytical sensors 36, such as a feedback system 32 or a pH adjustment supply. The analytical sensor 36 can be used to provide information on the characteristics and contents of the fluid in the system 1, or in more specific components, e.g., the biocontainer 10 and whether the contents of the biocontainer 10 are acceptable. If acceptable, an automatic release of the contents of the biocontainer 10 to a buffer solution application 46 can occur through the outlet line 16, a separate designated biocontainer outlet line, or automatic exchange of the biocontainer 10. Additionally, a pH sensor can be used to automatically introduce fluid from a pH adjustment supply into the system 1 through a secondary pH inlet line 38 as a way of balancing the pH of the fluid in the system 1 before or during circulation cycles. The analytical sensor(s) 36 can be used to assist in confirming the homogeneity of the solution, finalize the buffer solution or media preparation through pH adjustment, and other functions as will be appreciated by one skilled in the art.

During circulation, it can be advantageous to monitor the amount of pressure being exerted on various lines and components of the system 1. For example, the flow can be reduced in the dissolution line 24 if the pressure needs to be decreased in order to prevent damage to the buffer cartridge 26, or to facilitate a better dissolution of the solute powder in the buffer cartridge 26 with the fluid circulating through the dissolution line 24. To prevent an excess of pressure in the buffer cartridge 26 and the dissolution line 24, for example, the bypass line 28 can be used to divert at least some of the flow of fluid from the dissolution line 24 and bypass the cartridge 26. In the embodiment illustrated in FIG. 1, the bypass valve 23 can be provided and controlled by a controller to selectively open the bypass valve 23 to alleviate pressure on the dissolution line 24. The degree to which the bypass valve 23 is opened, or the degree to which the pumps 18a and 18b in the embodiments of FIGS. 2 and 3 are operating, can be adjusted by a controller to help divert at least some amount of fluid from the dissolution line 24 into the bypass line 28, which fluid is then joined with and recirculated with the fluid exiting the buffer cartridge 26. Therefore, the fluid that splits and enters the dissolution line 24 and the bypass line 28 is then rejoined and recirculated together, allowing pressure to be managed within the buffer cartridge 26 without seriously affecting the flow of fluid or fluid pressure in the system 1 as a whole. The pump 18 of FIG. 1 can also be utilized in embodiments to adjust pressure or flow of fluid.

To assist in managing the fluid pressure in the lines and components of the system, and to help enable an effective movement of solute from the buffer cartridge 26, a feedback system 32 can be implemented in the system 1. The feedback system 32 can help control the flow of fluid through the system 1 and specific components, e.g., the buffer cartridge 26. The feedback system 32 can selectively control the flow of fluid through the dissolution line 24, the bypass line 28, and other components of system 1. In embodiments, the feedback system 32 can control the flow of fluid based upon a pressure in the dissolution line 24 downstream of the buffer cartridge 26 between the cartridge 26 and the biocontainer 10. In other embodiments, the feedback system 32 can control the flow of fluid based upon a local pressure in a different location of the system 1.

In the embodiment of FIG. 1, the fluid can interact with a pressure sensor 34 en route to the pre-filter 6 in order for the pressure sensor to detect the pressure exerted by the flow of fluid circulating through the dissolution line 24. In the illustrated embodiment, the pressure sensor 34 is located downstream of the cartridge 26 between the cartridge 26 and the biocontainer 10, and more particularly between the cartridge 26 and the pre-filter 6. The pressure sensor 34 can be used in conjunction with other sensors or information available to the system in a feedback system 32 configured to regulate the pressure in the system 1, such as by controlling the state or degree to which clamp valve 22 is open. In embodiments, the feedback system 32 interacts with the pressure sensor 34 and at least one other pressure or flow sensors positioned on the system 1, such as one located at another location in the dissolution line 24, including, for example, upstream of the buffer cartridge 26, upstream or downstream of the upstream filter 20, downstream of the downstream filter 6, and/or one located in the bypass line 28. In embodiments, the feedback system 32 and the pressure sensor 34 can be part of the means for controlling the flow of fluid through the cartridge wherein the flow controlling means are configured to selectively control the flow of fluid through at least one of the dissolution line 24 and the bypass line 28 based upon a pressure in the dissolution line 24 downstream of the cartridge 26 between the cartridge 26 and the biocontainer 10.

The feedback system 32 can be configured to receive information from the pressure sensor 34 and any other sensor included in the system 1 and to transmit control signals to one or more other components, e.g., the pump 18, the dissolution valve 22, and/or the bypass valve 23. The feedback system 32 can be configured to automatically regulate the pump 18, the dissolution valve 22, and/or the bypass valve 23 to maintain the flow of fluid through the dissolution line 24 within a specified fluid pressure range. For example, the feedback system 32 may utilize actuators or other automatic controls to adjust the degree to which the clamp valves 22, 23 are opened or the degree to which the pump 18, and the pumps 18a, 18b of FIGS. 2 and 3 are operated. The feedback system 32 may also be configured to provide an indication to an operator of the system 1 of the operational position and status of clamp valves or pumps of the system 1, and an operator interface adapted to allow the operator to adjust one or more selected clamp valves or pumps. The feedback system 32 may also be in electronic communication with various components of the system 1, e.g., the clamp valves 22, 23 and the pump 18 or the pumps 18a, 18b, such that the feedback system 32 automatically controls the operation of pumps or clamp valves.

In embodiments, the pressure sensor 34 can be augmented, replaced by, or comprised of various means of evaluating pressure. For example, a mechanical or electronic, e.g., electromagnetic, flow sensor can be used instead of, in conjunction with, the pressure sensor 34. Moreover, a timing system, component, and/or components may be utilized to measure the flow rate of the fluid moving through the system 1, either in one position in the system 1 or at multiple positions along the system 1. Additionally, approximations based on volume of water, cross section of pipes, and fluid flow rate, or by measuring and determining the tension in the tubing for non-rigid plastic tubing can all be used separately or together instead of or in conjunction with the pressure sensor 34. Further, the feedback system 32 may be operable without use of an electric sensor or circuitry, such as through a pressure sensor 34 with spring-based valve management that is responsive in proportion to the pressure exerted on the spring-based valve.

Monitoring and controlling the pressure in the system 1, such as in the dissolution line 24 or the bypass line 28, can be advantageous. For example, if the dissolution valve 22 is an open position that is too great and/or the pump 18 is creating a flow of fluid that is excessive, an accumulation of pressure can occur in the buffer cartridge 26 caused by blockages of the powder, or in the filters 8, 20 as a result of excessive fluid pressure and/or solute blockages. Either of these blockages can impede or halt the flow of fluid through the circulation loop, reduce the efficiency of the system 1, lead to a stoppage of the entire buffer or media preparation process, and/or damage the lines 24, 28, connections, or other components of the system 1. Moreover, because the actual settings of the pressure and pump flow will vary depending on the kind of buffer that is added, e.g., the characteristics of its solubility, and the relative proportion of buffer to total liquid, the settings of various components such as clamp valves and pumps may change when preparing different buffer solutions. The pressure sensor 34 in combination with the feedback system 32 can therefore be used to follow a protocol with an appropriate progressive fluid flow profile through the buffer cartridge 26 and the system 1.

In the embodiment of FIG. 2, the system 1 includes two pumps 18a, 18b to regulate the flow of fluid through the system 1. The bypass pump 18a can operate on the bypass line 28, while the dissolution pump 18b can operate on the dissolution line 24. In the embodiment of FIG. 2, the dissolution pump 18a is in fluid communication with the upstream filter 8 and the pre-filter 6, and the pump 18b is in fluid communication with the buffer cartridge 26 and the downstream filter 8. The bypass pump 18a and the dissolution pump 18b may be respectively positioned in the bypass line 28 and the dissolution line 24 such that the pump 18a, 18b affects the flow of fluid respectively through the bypass line 28 and the dissolution line 24 to effectively act as a part of a flow control system and as part of the means for controlling the flow of fluid through the cartridge.

In the embodiment of FIG. 2, the bypass and dissolution pumps 18a, 18b can be used in lieu of, or in addition to, clamp valves respectively disposed in the bypass and dissolution lines 28, 24 as part of the flow controlling means configured to selectively control the flow of fluid through at least one of the dissolution line and the bypass line. In embodiments, the bypass and dissolution pumps 18a, 18b replace or function as a valve respectively controlling the flow of fluid through the bypass line 28 and the dissolution line 24. The bypass and dissolution pumps 18a, 18b act as a valve by either operating to effectively open the line relative to the other line in which the other pump is not operating (or operating at a lower rate) or not running to effectively close the line relative to the other line in which the other pump is operating (or operating at a higher rate). For example, the pumps 18a, 18b may be operated, or not operated, to exert a pressure or fluid flow rate that is equivalent to a pressure or fluid flow rate in the dissolution line 24 and the bypass line 28 that would result from a corresponding operation of the clamp valves 22, 23 of the embodiment of FIG. 1. Additionally, the bypass and dissolution pumps 18a, 18b can be configured such that fluid flows through the pumps 18a, 18b in a pass through mode without the pumps 18a, 18b being in operation, for example, and/or such that no fluid can pass through the pumps 18a, 18b when they are not in operation, for example. Moreover, the bypass pump 18a can be configured to operate in a different state than the dissolution pump 18b, e.g., the bypass pump 18a may be configured to replicate or replace the bypass valve 23, while the dissolution pump 18b is not, and vice-versa.

In the embodiment of FIG. 3, the bypass and dissolution clamp valves 23, 22 are used in conjunction with the bypass and dissolution pumps 18a, 18b as part of the means for controlling the flow of fluid through the cartridge 26. In embodiments, a different combination of clamp valves can be operable with a different combination of pumps to achieve effective flow control within the dissolution and bypass lines 24, 28. For example, while FIG. 3 shows the bypass clamp valve 23 following the bypass pump 18a and the dissolution clamp valve 22 following the dissolution pump 18b, the clamp valves 22, 23, may precede the pumps 18a, 18b. Moreover, the clamp valves may be operable and positioned as desired to fluidly isolate the pumps 18a, 18b from other components of the system 1. The system 1 may also include a bypass line 28 without a clamp valve 23, while the dissolution line 24 includes a clamp valve 22, and vice-versa.

The bypass and dissolution pumps 18a, 18b may be operated independently, or based upon the operation of an additional pump such as the pump 18 in the embodiment of FIG. 1 which is disposed upstream of the upstream junction 81 from which the bypass line 28 branches. For example, the bypass and dissolution pumps 18a, 18b, or an additional pump, may be operated such that the increase or decrease of the fluid pressure or speed exerted by one pump, e.g., pump 18, directly affects the fluid pressure or speed exerted by one or both of the other pump, e.g., pumps 18a, 18b. Moreover, either the pump 18a or the pump 18b, or an additional pump, can be configured to operate in any combination of independence or dependence with respect to each other. For example, the pump 18b may be configured to adjust fluid pressure or speed based on a fluid pressure or speed exerted by the pump 18a, while the pump 18a may not be configured to adjust fluid pressure or speed based on a fluid pressure or speed exerted by the pump 18b, and vice-versa.

The placement of the upstream filter 20 and downstream filter 8 can help achieve many advantages. For example, as shown diagrammatically in FIG. 2, the system 1 can be divided along a sterile line 40 that defines a sterile portion of the system 1 (on the right-hand side of the sterile line 40) and a non-sterile portion (on the left-hand side of the sterile line 40). The biocontainer 10 is located in the sterile portion of the system 1. The sterile portion of the circulation cycle can be created and maintained in the system 1 and the biocontainer 10 between the sterile filters 8, 20, which in the diagrammatic embodiment of FIG. 2 includes all components to the right of the sterile line 40. Being able to create a sterile environment in the portion of the circulation loop between the sterile filters 8, 20 allows for the buffer cartridge 26 to be either sterile or non-sterile without affecting the sterility of the biocontainer 10. Additionally, having a bypass line 28 run parallel with the buffer cartridge 26 in the dissolution line 24 alleviates the risk of blockage accruing at some point in the circulation loop due to an over presence or accumulation of buffer solute, such as in the pre-filter 6 or the downstream filter 8.

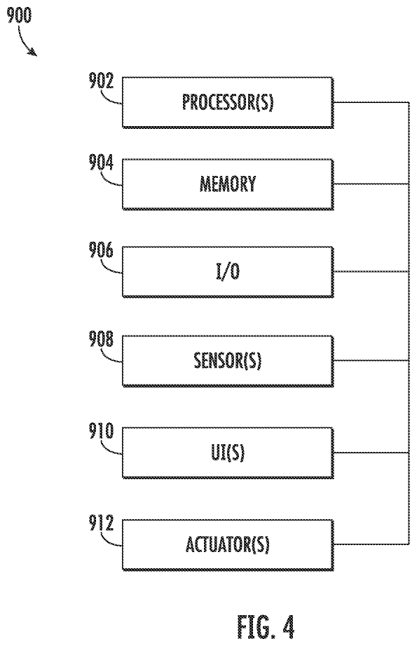

Referring to FIG. 4, the system can include a suitable controller 900 for use with the feedback system 32, the pressure sensor 34, the analysis sensor 36, and any other suitable sensor desired to be used. The controller can be configured to control the operation of one or more valves and/or one or more pumps based upon a sensor signal received from a sensor placed in communicative arrangement therewith. In embodiments, any suitable commercially-available controller can be used. In embodiments, the controller 900 can include one or more processors 902, a memory 904, one or more input/output devices 906, one or more sensors 908, one or more user interfaces 910, and one or more actuators 912. The controller 900 can be representative of each controller system disclosed herein.

The processors 902 can include one or more distinct processors, each having one or more cores. Each of the distinct processors can have the same or different structure. The processors 902 can include one or more central processing units (CPUs), one or more graphics processing units (GPUs), circuitry (e.g., application specific integrated circuits (ASICs)), digital signal processors (DSPs), and the like. The processors 902 can be mounted to a common substrate or to multiple different substrates.

The processors 902 are configured to perform a certain function, method, or operation (e.g., are configured to provide for performance of a function, method, or operation) at least when one of the one or more of the distinct processors is capable of performing operations embodying the function, method, or operation. The processors 902 can perform operations embodying the function, method, or operation by, for example, executing code (e.g., interpreting scripts) stored on the memory 904 and/or trafficking data through one or more ASICs. The processors 902, and thus the controller 900, can be configured to perform, automatically, any and all functions, methods, and operations disclosed herein. Therefore, the controller 900 can be configured to implement any of (e.g., all of) the protocols, devices, mechanisms, systems, and methods described herein.

For example, when the present disclosure states that a method or device performs task “X” (or that task “X” is performed), such a statement should be understood to disclose that the controller 900 can be configured to perform task “X.” The controller 900 is configured to perform a function, method, or operation at least when the processors 902 are configured to do the same.

The memory 904 can include volatile memory, non-volatile memory, and any other medium capable of storing data. Each of the volatile memory, non-volatile memory, and any other type of memory can include multiple different memory devices, located at multiple distinct locations and each having a different structure. The memory 904 can include remotely hosted (e.g., cloud) storage.

Examples of the memory 904 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in the memory 904.

Input-output devices 906 can include any component for trafficking data such as ports, antennas (i.e., transceivers), printed conductive paths, and the like. Input-output devices 906 can enable wired communication via USB®, DisplayPort®, HDMI®, Ethernet, and the like. Input-output devices 906 can enable electronic, optical, magnetic, and holographic, communication with suitable memory 906. Input-output devices 906 can enable wireless communication via WiFi®, Bluetooth®, cellular (e.g., LTE®, CDMA®, GSM®, WiMax®, NFC®), GPS, and the like. Input-output devices 906 can include wired and/or wireless communication pathways.

Sensors 908, e.g., the pressure sensor 34, can capture physical measurements of environment and report the same to the processors 902. User interface 910 can include displays, physical buttons, speakers, microphones, keyboards, and the like. Actuators 912 can enable processors 902 to control mechanical forces.

The controller 900 can comprise a distributed processing system. For example, some components of the processing system 900 can reside in a remote hosted network service (e.g., a cloud computing environment) while other components of the processing system 900 can reside in a local computing system. The controller 900 can have a modular design where certain modules include a plurality of the features/functions shown in FIG. 4. For example, I/O modules can include volatile memory and one or more processors. As another example, individual processor modules can include read-only-memory and/or local caches.

Embodiments of a system constructed according to principles of the present disclosure can function with the relative positioning of many of the various components changed with respect to each other, e.g., the positioning of the pump 18, the bypass line 28 and the dissolution line 24, and even the repositioning of the sterile filters 8, 20 with respect to other components or each other. Embodiments of a system constructed according to principles of the present disclosure can function with the addition or omission of various components of the system 1, such as by adding additional clamp valves or pumps to the system 1 to regulate flow on various fluid lines differently, or by removing the pre-filter 6 or integrating the pre-filter with the downstream filter 8, for example. The system 1 may also be compatible with various other systems and processes, and may have additional lines and/or fluid connections to work with those other systems. Additional flow sensors can be integrated into the system 1 in multiple locations for better process control, and can even be integrated into other existing components, such as the pump 18. Additional vents can also be integrated into the system 1 to be selectively opened or closed in the event air or other matter is accumulating or becoming trapped in certain locations in the system 1, further assisting in managing the sterility and/or pressure levels of the system 1. Braided tubes can also be utilized in areas of the system 1 that experience sufficient amounts of pressure, such as the dissolution line 24.

The structure and material of the components of the system 1 can be selected according to various advantages and desires. For example, all components can be reusable, such as steel or other rigid elements, or single use, or supported by hardware, depending on the cost, sanitation, and mobility considerations of the buffer or media preparation process. If the fluid deposited in the biocontainer 10 does not need to be sterile, the sterile filters 8, 20 can be removed to reduce unnecessary costs. Moreover, certain components or lines of the system 1 can be transparent to provide better visual feedback of the dissolution circulation, such as tracking the rate of solute dissolution or air/particulate flow in the fluid.

A process for preparing a buffer dissolution following principles of the present disclosure can be used with any one of the systems of FIGS. 1-3. In embodiments, the buffer cartridge 26 can be filled with the solid material 44, e.g., a suitable buffer solute in powder or pellet form to be dissolved. Pre-circulation can selectively occur where the clamp valves 4, 12, 23, and 30 are opened and fluid circulates from the biocontainer 10 through the lines of the system 1, except the dissolution line 24, and back into the biocontainer 10. The clamp valve 4 is opened, and the clamp valves 14, 30 are closed. The upstream filter 8 is vented and the biocontainer 10 is filled slightly. The clamp valve 30 is then opened. The biocontainer 10 is filled, and both the pre-filter 6 and the downstream filter 8 are vented. After the biocontainer 10 is filled, the clamp valve 4 is closed.

A flow of fluid is initiated in the bypass line 28, either by opening the clamp valve 23 to a desired amount or, for example, by operating a pump 18a associated with the bypass line 28. The buffer cartridge 26 is introduced to the system 1 as in FIG. 2, and a flow of fluid is initiated in the dissolution line 24, either by opening the clamp valve 22 to a desired amount as in the embodiment of FIG. 1 or, for example, by operating a pump 18 associated with the dissolution line 24 as in FIG. 2, or a combination of either or both as in the embodiment of FIG. 3. The flow in the dissolution line 24 can be in parallel to the flow in the bypass line 28, and fluid can be diverted between the dissolution line 24 and the bypass line 28 as desired. Throughout this process, the fluid going through the buffer cartridge 26 is taking up the substances 44, e.g., buffer solute or powder, and flushing at the entrance of the pre-filter 6. The flow of fluid through the bypass line 28 is coming into contact with the buffer solute pushed from the buffer cartridge 26 just after the clamp valve 30, and accumulating in front of the pre-filter 6. The flow of fluid through the bypass line 28 can facilitate the dissolution of the buffer solute into the solvent or fluid phase, which allows the flow of solution through both the pre-filter 6 and the downstream filter 8. In embodiments, the pressure sensor 34 is disposed upstream of the pre-filter 6 and is configured to cooperate with the feedback system 32 to allow for the control of the flow of fluid through the dissolution line 24 so that not too much solute is flushed from the buffer cartridge 26, which could lead to a potential blockage and an abrupt ending of the process.

In embodiments, the fluid pressure in the dissolution line 24 is also monitored. If pressure in the dissolution line 24 is below a predetermined threshold or decreases above a predetermined rate, the flow of fluid in the dissolution line 24 can be increased. If the pressure in the dissolution line 24 is above a predetermined threshold or increases above a predetermined rate, the flow of fluid in the dissolution line 24 can be reduced. In embodiments. The controller 900 cooperates with the pressure sensor 34 to monitor and adjust the flow of fluid either autonomously or in conjunction with other systems or operators. Moreover, the monitoring of pressure in the dissolution line 24 can be achieved indirectly, such as by monitoring other portions of the system 1 and adjusting fluid flows and the pressures in system 1 to achieve the same effect as monitoring the dissolution line 24. Once the clamp valve 22 is completely open and the maximum flow through the dissolution line 24 and the buffer cartridge 26 is achieved, the process can be continued until the buffer solute has been fully and completely dissolved. In embodiments, the bypass valve 23 can be progressively closed either once the dissolution valve 22 is fully open or as the dissolution valve 22 moves over a range of travel from a fully closed position to a fully open position.

Once sensor signal values collected from the analysis sensors 36 are constant (or within a predetermined range), pH adjustment can begin for final preparation of the buffer solution. Once all sensor signal values are constant (or within a predetermined range) after pH adjustment, the buffer preparation in the biocontainer 10 can be transferred to the next processing steps 48 for the batch 46, either by modular removal or draining the biocontainer 10 via the outlet line 16 by opening the clamp valve 14. The pre-filter 6 can be vented to allow air entry into the pre-filter 6. The pump 18 or the pumps 18a, 18b can also be actuated in the opposite direction until the lines of the system 1 and the biocontainer 10 are empty.

In an embodiment, in an initial step, the buffer cartridge 26 can be filled with the buffer solute to be dissolved, the inlet and bypass valves 4, 23 are opened, and the outlet, dissolution, and inlet valves 14, 22, 30 are closed. After a predetermined period of time has elapsed from the initial step, the inlet valve 30 is opened, the upstream filter 20 is vented, and the biocontainer 10 is filled slightly to an initial volume, and the bypass valve 23 is closed. After a predetermined period of time has elapsed after the initial step, the biocontainer 10 is filled from the initial volume to a greater, target volume, and both the pre-filter 6 and the downstream filter 8 are vented. After a predetermined period of time has elapsed from the initial step, the clamp valve 23 is opened, and the pump 18 is started.

The fluid pressure in the dissolution line 24 is then monitored. When pressure in the dissolution line 24 decreases below a threshold, the flow of fluid in the dissolution line 24 can be increased. The pressure in the system 1 can be monitored and the dissolution and bypass valves 22, 23 can be adjusted as needed for a predetermined period of time. The system 1 can be drained by opening the drain valve 14.

In other embodiments, the pump 18 can be positioned at a different location in the hydraulic circuit, as desired. In embodiments, an additional pump, stronger or more reinforced tubing, and/or or better or larger filters can be used to increase the efficiency of the embodiments of the present disclosure and reduce the time necessary to complete a preparation or increase the quality or quantity of the preparation within the same time period. In embodiments, percussive force can be exerted on various components of the system 1 to help prevent clogging of passageways caused by buildup of solute.

In embodiments of a method of preparing a solution following principles of the present disclosure, any suitable embodiment of a dissolution system constructed according to principles discussed herein can be used. In embodiments, a method of preparing a solution following principles of the present disclosure comprises using a recirculation system with a dissolution line, a bypass line, and a flow control system following principles of the present disclosure.

In one embodiment, a method of preparing a buffer solution includes fluidly coupling a buffer cartridge in a circulation loop formed by a dissolution line. The dissolution line fluidly couples in the circulation loop a biocontainer, a pump adapted to discharge a flow of fluid therefrom in a circulation direction, the buffer cartridge, an upstream filter disposed upstream of the buffer cartridge relative to the circulation direction between the biocontainer and the buffer cartridge, and a downstream filter disposed upstream of the buffer cartridge relative to the circulation direction between the buffer cartridge and the biocontainer. The buffer cartridge contains an amount of a buffer solute.

A flow of fluid is circulated through the circulation loop to entrain at least a portion of the buffer solute from the buffer cartridge into the flow of fluid. In embodiments of a method of preparing a buffer solution, circulating the flow of fluid through the circulation loop includes controlling the flow of fluid through the buffer cartridge based upon a pressure in the dissolution line downstream of the buffer cartridge between the buffer cartridge and the biocontainer.

In embodiments of a method of preparing a buffer solution, the buffer solute comprises a powder. The method can further include, before fluidly coupling the buffer cartridge in the circulation loop, filling the buffer cartridge with the amount of buffer solute in a buffer transfer room.

In embodiments of a method of preparing a buffer solution, the dissolution line includes an upstream junction and a downstream junction. The upstream junction is disposed upstream of the buffer cartridge relative to the circulation direction between the upstream filter and the buffer cartridge, and the downstream junction is disposed downstream of the buffer cartridge relative to the circulation direction between the buffer cartridge and the downstream filter. The method can further include diverting at least a portion of the flow of fluid from the dissolution line into a bypass line. The bypass line is in fluid communication with the dissolution line at the upstream junction and the downstream junction such that the bypass line is in parallel relationship with the cartridge.

In at least some of such embodiments, diverting at least a portion of the flow of fluid from the dissolution line into a bypass line includes adjusting the amount of the flow of fluid diverted into the bypass line based upon a pressure in the dissolution line downstream of the cartridge between the cartridge and the biocontainer. In at least some of such embodiments, adjusting the amount of the flow of fluid diverted into the bypass line based upon the pressure in the dissolution line downstream of the cartridge between the cartridge and the biocontainer includes adjusting the speed of the pump in the dissolution line in inverse relationship to the pressure.

In embodiments of a method of preparing a buffer solution, the method can include entraining substantially all of the buffer solute in the buffer cartridge into the flow of fluid. The flow of fluid is circulated through the circulation loop to substantially dissolve the buffer solute to form a buffer solution. A pH value of the buffer solution is sensed using a sensor. The pH of the buffer solution is adjusted to a target pH range by introducing a pH adjustment supply into the buffer solution based upon the sensed pH value.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A dissolution system comprising:
a biocontainer, the biocontainer defining a container inlet, a container outlet, and a storage volume, the container inlet and outlet in communication with the storage volume, and the storage volume configured to hold a supply of fluid;
a pump, the pump in fluid communication with the storage volume of the biocontainer, the pump adapted to receive the supply of fluid from the container outlet of the biocontainer and to discharge a flow of fluid therefrom in a circulation direction to the container inlet;
a cartridge, the cartridge defining a cartridge inlet, a cartridge outlet, and a storage chamber, the cartridge inlet and outlet in communication with the storage chamber, the storage chamber configured to hold an amount of solute for dissolution into the supply of fluid, the storage chamber in fluid communication with the pump via the cartridge inlet to receive the flow of fluid therefrom, the cartridge inlet, the storage chamber, and the cartridge outlet configured such that the flow of fluid is directed from the cartridge inlet through storage chamber and out the cartridge outlet to flow past the amount of solute in the storage chamber, the cartridge outlet in fluid communication with the container inlet;
an upstream filter, the upstream filter in fluid communication with the biocontainer and the cartridge such that the upstream filter is interposed between the container outlet of the biocontainer and the cartridge inlet of the cartridge upstream of the cartridge relative to the circulation direction;
a downstream filter, the downstream filter in fluid communication with the cartridge and the biocontainer such that the downstream filter is interposed between the cartridge outlet of the cartridge and the container inlet of the biocontainer downstream of the cartridge relative to the circulation direction;
a dissolution line, the dissolution line fluidly coupling the biocontainer, the pump, the cartridge, the upstream filter, and the downstream filter in a circulation loop.

2. The dissolution system according to claim 1, further comprising:
a pre-filter, the pre-filter in fluid communication with the cartridge and the downstream filter such that the pre-filter is interposed between the cartridge and the downstream filter, the downstream filter having a first interior volume and the pre-filter having a second interior volume, the second interior volume being greater than the first interior volume.

3. The dissolution system according to claim 1, further comprising:
a mixer, the mixer in fluid communication with the cartridge and the downstream filter, such that the mixer is interposed between the cartridge and the downstream filter.

4. The dissolution system according to claim 3, wherein the mixer comprises a static mixer.

5. The dissolution system according to claim 1, further comprising:
a valve, the valve adapted to selectively control the flow of fluid through the cartridge based upon a pressure in the dissolution line downstream of the cartridge between the cartridge and the biocontainer.

6. The dissolution system according to claim 1, wherein the dissolution line includes an upstream junction and a downstream junction, the upstream junction being disposed upstream of the cartridge relative to the circulation direction between the upstream filter and the cartridge inlet of the cartridge, and the downstream junction being disposed downstream of the cartridge relative to the circulation direction between the cartridge outlet of the cartridge and the downstream filter, the dissolution system further comprising:
a bypass line, the bypass line being in fluid communication with the dissolution line at the upstream junction and the downstream junction such that the bypass line is in parallel relationship with the cartridge.

7. The dissolution system according to claim 6, further comprising:
a flow control system, the flow control system configured to selectively control the flow of fluid through at least one of the dissolution line and the bypass line.

8. The dissolution system according to claim 7, wherein the pump comprises a dissolution pump, the dissolution pump disposed in the dissolution line between the upstream junction and the cartridge inlet, and wherein the flow control system includes a bypass pump and a pressure sensor, the bypass pump disposed in the bypass line, and the pressure sensor disposed in the dissolution line between the cartridge outlet and the container inlet, the pressure sensor configured to generate a pressure signal corresponding to a pressure sensed in the dissolution line by the pressure sensor, and at least one of the bypass pump and the dissolution pump adapted to operate based upon the pressure signal.

9. The dissolution system according to claim 7, wherein the pump is disposed in the dissolution line between the container outlet and the upstream junction, and wherein the flow control system includes a dissolution valve, a bypass valve, and a pressure sensor, the dissolution valve disposed in the dissolution line between the upstream junction and the cartridge inlet, the bypass valve disposed in the bypass line, and the pressure sensor disposed in the dissolution line between the cartridge outlet and the container inlet, the pressure sensor configured to generate a pressure signal corresponding to a pressure sensed in the dissolution line by the pressure sensor, and at least one of the dissolution valve and the bypass valve adapted to operate based upon the pressure signal.

10. The dissolution system according to claim 9, further comprising:
a solution characteristic sensor, the solution characteristic sensor disposed in the dissolution line, the solution characteristic sensor configured to generate a characteristic signal corresponding to a value of a solution characteristic sensed in the dissolution line by the solution characteristic sensor.

11. The dissolution system according to claim 10, wherein the solution characteristic sensor comprises a pH sensor, and the characteristic signal comprises a pH signal, the system further comprising:
a pH adjustment line and a pH adjustment valve, the pH adjustment line in fluid communication with the dissolution line and adapted to deliver a supply of pH-adjusting fluid to the dissolution line, the pH adjustment valve operable to selectively occlude the pH adjustment line to interrupt the flow of the supply of pH-adjusting fluid to the dissolution line, the pH adjustment valve adapted to operate based upon the pH signal.

12. The dissolution system according to claim 11, wherein the pH adjustment line is in fluid communication with the dissolution line via the bypass line.

13. A dissolution system comprising:
a dissolution line, the dissolution line fluidly coupling a biocontainer, a pump, a cartridge, an upstream filter, and a downstream filter in a circulation loop, the biocontainer configured to hold a supply of fluid,
the pump adapted to receive the supply of fluid from the biocontainer and to discharge a flow of fluid therefrom in a circulation direction;
the cartridge configured to hold an amount of solute for dissolution into the supply of fluid, the cartridge in fluid communication with the pump to receive the flow of fluid therefrom and to pass the flow of fluid therethrough,
the upstream filter in fluid communication with the biocontainer and the cartridge such that the upstream filter is interposed between the biocontainer and the cartridge upstream of the cartridge relative to the circulation direction, and
the downstream filter in fluid communication with the cartridge and the biocontainer such that the downstream filter is interposed between the cartridge and the biocontainer downstream of the cartridge relative to the circulation direction,
wherein the dissolution line includes an upstream junction and a downstream junction, the upstream junction being disposed upstream of the cartridge relative to the circulation direction between the upstream filter and the cartridge, and the downstream junction being disposed downstream of the cartridge relative to the circulation direction between the cartridge and the downstream filter;
a bypass line, the bypass line being in fluid communication with the dissolution line at the upstream junction and the downstream junction such that the bypass line is in parallel relationship with the cartridge;
means for controlling the flow of fluid through the cartridge, the flow controlling means configured to selectively control the flow of fluid through at least one of the dissolution line and the bypass line based upon a pressure in the dissolution line downstream of the cartridge between the cartridge and the biocontainer.

14. A method of preparing a buffer solution, the method comprising:
fluidly coupling a buffer cartridge in a circulation loop formed by a dissolution line, the dissolution line fluidly coupling in the circulation loop a biocontainer, a pump adapted to discharge a flow of fluid therefrom in a circulation direction, the buffer cartridge, an upstream filter disposed upstream of the buffer cartridge relative to the circulation direction between the biocontainer and the buffer cartridge, and a downstream filter disposed upstream of the buffer cartridge relative to the circulation direction between the buffer cartridge and the biocontainer, the buffer cartridge containing an amount of a buffer solute;
circulating a flow of fluid through the circulation loop to entrain at least a portion of the buffer solute from the buffer cartridge into the flow of fluid.

15. The method according to claim 14, further comprising:
before fluidly coupling the buffer cartridge in the circulation loop, filling the buffer cartridge with the amount of buffer solute in a buffer transfer room, the buffer solute comprising a powder.

16. The method according to claim 14, wherein circulating the flow of fluid through the circulation loop includes controlling the flow of fluid through the buffer cartridge based upon a pressure in the dissolution line downstream of the buffer cartridge between the buffer cartridge and the biocontainer.

17. The method according to claim 14, wherein the dissolution line includes an upstream junction and a downstream junction, the upstream junction being disposed upstream of the buffer cartridge relative to the circulation direction between the upstream filter and the buffer cartridge, and the downstream junction being disposed downstream of the buffer cartridge relative to the circulation direction between the buffer cartridge and the downstream filter, the method further comprising:
diverting at least a portion of the flow of fluid from the dissolution line into a bypass line, the bypass line being in fluid communication with the dissolution line at the upstream junction and the downstream junction such that the bypass line is in parallel relationship with the buffer cartridge.

18. The method according to claim 17, wherein diverting at least a portion of the flow of fluid from the dissolution line into a bypass line includes adjusting the amount of the flow of fluid diverted into the bypass line based upon a pressure in the dissolution line downstream of the buffer cartridge between the buffer cartridge and the biocontainer.

19. The method according to claim 18, wherein adjusting the amount of the flow of fluid diverted into the bypass line based upon the pressure in the dissolution line downstream of the buffer cartridge between the buffer cartridge and the biocontainer includes adjusting the speed of the pump in the dissolution line in inverse relationship to the pressure.

20. The method according to claim 14, further comprising:
entraining substantially all of the buffer solute in the buffer cartridge into the flow of fluid;
circulating the flow of fluid through the circulation loop to substantially dissolve the buffer solute to form a buffer solution;
sensing, using a sensor, a pH value of the buffer solution;
adjusting the pH of the buffer solution to a target pH range by introducing a pH adjustment supply into the buffer solution based upon the sensed pH value.
